(19) United States
(12) Patent Application Publication
(10) Pub. No.: US 2005/0031579 A1
(43) Pub. Date: Feb. 10, 2005

(54) Polymer encapsulation of adenoviruses

(75) Inventor: Thomas Schluep, La Crescenta, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Canji, Inc., San Diego, CA (US)

(21) Appl. No.: 10/870,813

(22) Filed: Jun. 16, 2004

Related U.S. Application Data
(60) Provisional application No. 60/484,060, filed on Jun. 30, 2003.

(51) Int. Cl. A61K 39/23; A61K 39/235

(52) U.S. Cl. 424/78.27; 424/233.1

(57) ABSTRACT

The present invention provides a copolymer that noncovalently encapsulates an adenovirus while improving its delivery and resulting expression from the viral genome. It has now been discovered that a copolymer of a cationic polymer, such as PEI, polylysine, DEAE-Dextran, and derivatives thereof, and a nonionic polymer, such as PEG and derivatives thereof, can improve both delivery and transgene expression of the adenovirus in cells. The complex of the invention provides an easy-to-produce material that is therapeutically more effective than an unencapsulated adenovirus.

### CROSS-REFERENCE TO RELATED APPLICATION

This application claims the benefit of U.S. Ser. No. 60/484,060, filed Jun. 30, 2003, the disclosure of which is incorporated herein by reference.

### BACKGROUND OF THE INVENTION

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Recombinant adenovirus is used extensively as a vector in gene therapy due to its ability to deliver genes into a wide variety of proliferating and non-proliferating cells. Systemic delivery of adenovirus, however, faces several hurdles such as short blood half life (Morrissey et al., Toxicol Sci. 65: 266-275 (2002); Alemany et al., J Gen Virol. 81: 2605-2609 (2000)), elimination by the reticuloendothelial system (RES) (Ziegler et al., Hum Gene Ther. 13: 935-945 (2002); Tao et al., Mol Ther. 3: 28-35 (2001)), elicitation of an innate immune response (Zhang et al., Mol Ther. 3: 697-707 (2001); Schnell et al., Mol Ther. 3: 708-722 (2001)), neutralization by pre-existing antibodies (Rahman et al., Mol Ther. 3: 768-778 (2001)), and a natural tropism of adenoviral subtypes for certain tissues and cell types (Bergelson, Biochem Pharmacol. 57: 975-979 (1999)). Additionally, because adenovirus elicits a strong humoral immune response, repeat administrations are difficult (Rahman et al., Mol Ther. 3: 768-778 (2001)).

To circumvent some of these issues, others have used covalent attachment of hydrophilic polymers such as PEG (O'Riordan et al., Hum Gene Ther. 10: 1349-1358 (1999); Croyle et al., Hum Gene Ther. 11: 1713-1722 (2000)) or pHPMA (Fisher et al., Gene Ther. 8: 341-348 (2001)) to the adenovirus capsid, changed the virus surface by genetically re-engineering the fiber (Hidaka et al., J Clin Invest. 103: 579-587 (1999)), or encapsulated the rAd into liposomes (Yotnda et al., Mol Ther. 5: 233-241 (2002)). Strategies modifying the adenovirus surface with hydrophilic polymers (e.g., PEG) are based on well established findings that covalent modification of proteins and enzymes with PEG improves their therapeutic efficacy with enhanced circulation half life, reduced immunogenicity, enhanced solubility, and suitable in vivo bioactivity (Harris et al., Clin Pharmacokinet. 40: 539-551 (2001); Wang et al., Adv Drug Deliv Rev. 54: 547-570 (2002)).

Covalent attachment of adenovirus to an encapsulating polymer requires an additional purification step and subsequently lowers the chemical yield. In addition, infectivity of adenovirus is typically reduced when PEG is covalently attached (O'Riordan et al., Hum Gene Ther. 10: 1349-1358 (1999); Croyle et al., Hum Gene Ther. 11: 1713-1722 (2000)). The present invention addresses this and other problems.

### BRIEF SUMMARY OF THE INVENTION

The present invention provides for noncovalent complexes of copolymers and adenoviruses. The copolymer, which is a combination of a cationic polymer, such as PEI, polylysine, DEAE-Dextran, and derivatives thereof, and a nonionic polymer, such as PEG and derivatives thereof, can improve both delivery and transgene expression of the adenovirus in cells. The complex of the invention provides an easy-to-produce material that is therapeutically more effective than an unencapsulated adenovirus.

In a first aspect, the invention provides a complex comprising an adenovirus noncovalently complexed to a copolymer. The copolymer comprises a structure according to Formula I: 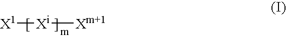
in which m is an integer from 1 to 1,000. The symbol i is an integer from 2 to m and denotes the position of Xⁱ. The symbols X¹, Xⁱ, and X^{m+1} are independently selected monomers, wherein (i) said monomers comprise an amine selected from secondary amines and tertiary amines; and (ii) at least one of said monomers comprise Q. Q is a structure selected from Formula IIa and Formula IIb: 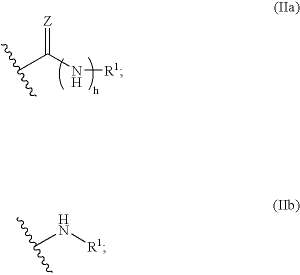
in which Z is selected from the group consisting of O and NH. The symbol h is an integer from 0 to 1. The symbol R¹ comprises a polyalkylene glycol moiety. The copolymers of the invention are also free of cross-polymerization, and, at physiological pH, at least one of the nitrogen atoms in the copolymer is positively charged.

In a second aspect, the copolymers of Formula I are involved in a method of preparing a noncovalently complexed adenovirus copolymer complex.

In a third aspect, the copolymers of Formula I are involved in a method of introducing an adenovirus into a cell. In this method, (a) an adenovirus is noncovalently contacted to a copolymer, and (b) the complex is contacted to a cell.

In a fourth aspect, the present invention provides a physiological formulation comprising: (a) a copolymer of Formula I; (b) an adenovirus, which forms a noncovalent complex with the adenovirus; and (c) a physiologically acceptable excipient.

In a fifth aspect, the present invention provides a kit comprising a copolymer of Formula I and an adenovirus, wherein the copolymer and adenovirus are noncovalently attached.

Other aspects and embodiments of the present invention will be apparent from the detailed description that follows.

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic synthesis, analytical chemistry, and nucleic acid chemistry and hybridization described below are those well known and commonly employed in the art. The techniques and procedures are generally performed according to conventional methods in the art and various general references (see generally, Knipe et al. FIELDS VIROLOGY, 4th ed. (2001) Lippincott, Williams, and Wilkins, Philadelphia, Pa., which is incorporated herein by reference), which are provided throughout this document. Standard techniques, or modifications thereof, are used for chemical syntheses and chemical analyses.

“Nucleic acid” refers to deoxyribonucleotides or ribonucleotides and polymers thereof in either single- or double-stranded form. The term encompasses the terms gene, cDNA, mRNA, oligonucleotide, and polynucleotide. The term also encompasses synthetic, naturally occurring, and non-naturally occurring nucleotide analogs with modified backbone residues or linkages. These nucleotide analogs have similar binding properties as the reference nucleic acid, or are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The term “contacting a cell” refers to the internalization of an adenovirus or complex of the invention into the cell. This term encompasses, for example, intravenous or oral administration of the virus or complex which results in its internalization into the cell.

The term “adenovirus” generally comprises a polynucleotide comprising all or a portion of an adenovirus genome. “Adenovirus” refers collectively to animal adenoviruses of the genus mastadenovirus including, but not limited to, human, bovine, ovine, equine, canine, porcine, murine and simian adenovirus subgenera. In particular, human adenoviruses include the A-F subgenera as well as the individual serotypes thereof, the individual serotypes and A-F subgenera including, but not limited to, human adenovirus types 1, 2, 3, 4, 4a, 5, 6, 7, 8, 9, 10, 11 (Ad11A and Ad 11P), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. The bovine adenoviruses useful in the invention include, but are not limited to, bovine adenovirus types 1, 2, 3, 4, 7, and 10. Canine adenoviruses include but are not limited to canine types 1 (strains CLL, Glaxo, RI261, Utrect, Toronto 26-61) and 2. Equine adenoviruses of interest include, but are not limited to, equine types 1 and 2 and porcine adenoviruses of interest include, for example, porcine types 3 and 4. “Adenovirus” also refers collectively to recombinant adenoviruses, such as those produced from the deletion, insertion, or mutation of nucleic acids. The recombinant adenoviruses can also be produced from the linking of DNA from different serotypes or subgenera.

The term “noncovalent”, as used herein, means the binding of substances via ionic bonding, electrostatic interactions, hydrogen bonding, hydrophilic-hydrophilic interactions, hydrophobic-hydrophobic interactions, van der Waals interactions and combinations thereof.

The term “polymer”, as used herein, refers to natural and synthetic compounds of usually high molecular weight consisting of up to millions of repeated linked monomers. Each monomer is a relatively light and simple molecule.

The term “homopolymer”, as used herein, refers to a polymer derived from a single type of monomer.

The term “copolymer”, as used herein, refers to a polymer produced by the simultaneous polymerization of two or more dissimilar monomers.

The term “cross-polymerization”, as used herein, refers to the covalent attachment of two or more polyalkylene imine moieties to opposing ends of a polyalkylene glycol molecule.

Where substituent groups are specified by their conventional chemical formulas, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Thus, a cycloalkyl or heterocycloalkyl include saturated and unsaturated ring linkages. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like:

As used herein, the term “polyalkylene glycol” refers to polyethylene glycol, polypropylene glycol, polybutylene glycol, and derivatives thereof. An exemplary embodiment of a polyalkylene glycol derivative is adipate dihydrazide-methyoxy-polyethylene glycol. Other exemplary embodiments are listed in Shearwater Corporation's catalog “Polyethylene Glycol and Derivatives for Biomedical Applications” (2001).

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

Each of the above terms (e.g., “alkyl,” “heteroalkyl,” “aryl” and “heteroaryl”) are meant to include both substituted and unsubstituted forms of the indicated radical. Exemplary substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such a radical. R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are independently selected from hydrogen, alkyl, heteroalkyl, aryl and heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —CRR′)ₙ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds that are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment.

The term “ring” as used herein means an encircling arrangement of atoms optionally having heteroatoms within the arrangement. A ring includes aromatic and non-aromatic moieties such as substituted or unsubstituted cycloalkyl, substituted or unsubstituted heterocycloalkyl, substituted or unsubstituted aryl and substituted or unsubstituted heteroaryl. The number of atoms in a ring are typically defined by the number of members in the ring. For example, a “5- to 7-membered ring” means there are 5-7 atoms in the encircling arrangement. Each member is optionally a heteroatom. Thus, the term “5- to 7-membered ring” includes, for example pyridinyl, piperidinyl and thiazolyl rings. Rings are typically drawn with a single explicit substituent within parentheses having a subscript letter. The subscript letter typically represents a set of integers, such as 1-10. The integers represent the number of ring substituents wherein each substituent is optionally different. For example, for the substituent (R¹)ₛ, where s is 2, the ring may be substituted with a substituted or unsubstituted alkyl and a substituted or unsubstituted heteroalkyl.

The term “poly” as used herein means at least 2. For example, a polyvalent metal ion is a metal ion having a valency of at least 2.

“Moiety” refers to the radical of a molecule that is attached to another structure.

The symbol 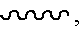
whether utilized as a bond or displayed perpendicular to a bond, indicates the point at which the displayed moiety is attached to the remainder of the molecule.

Certain compounds of the present invention possess asymmetric carbon atoms (optical centers) or double bonds; the racemates, diastereomers, geometric isomers and individual isomers are encompassed within the scope of the present invention.

### BRIEF DESCRIPTION OF THE DRAWINGS

FIG. 1: Schematic outline of the synthesis of PEI-mPEG copolymer. Branched polyethyleneimine (average x=2, y=1) with a molecular weight of 25 kDa was reacted with mPEG-SPA (5 kDa) in 133 mM borate, 100 mM sodium chloride, 0.7 mM EDTA, pH 8.4.

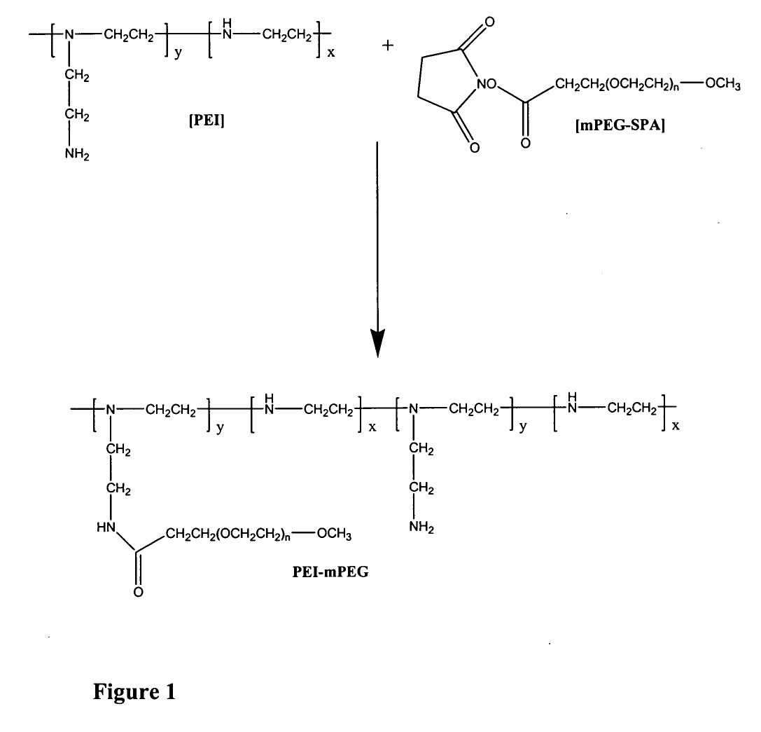

FIG. 2: Resource-Q anion exchange chromatograms of recombinant adenovirus (panel A) and adenovirus encapsulated with PEI-MPEG (panel B, encapsulation ratio C). The graphs show absorbance at 260 nm as a function of time. The insets show the absorbance spectra of the adenovirus peak (panel A, RT=10.37 minutes) and of the encapsulated adenovirus peak in the flow through (panel B, RT=0.64 minutes). Chromatographic conditions were as follows: Flow rate: 1 mL/minute, buffer A: 50 mM Hepes, pH 7.5, buffer B: Buffer A+1.5 M NaCl, gradient: 20% B to 40% B in 10 minutes.

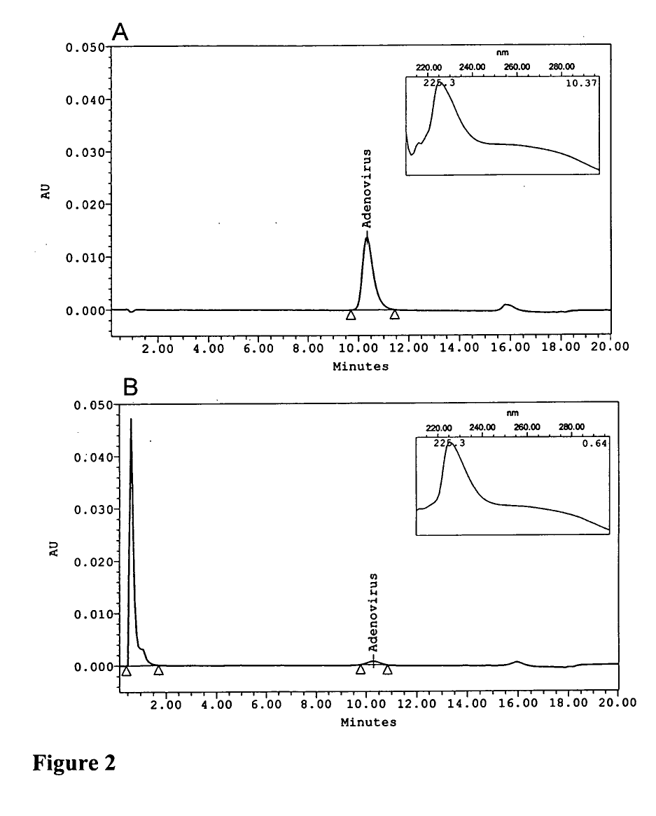

FIG. 3: In vitro infectivity and β-galactosidase expression of recombinant adenovirus and adenovirus encapsulated with PEI-mPEG. Cell lines used were T24 bladder carcinoma (white bars) and A549 lung carcinoma (black bars). Recombinant adenovirus expressing GFP (panel A) or β-Galactosidase (panel B) was encapsulated with PEI-mPEG using three different ratios of polymer to virus particles (rAd(enc) A, rAd(enc) B, and rAd(enc) C) as described in Table 1. Also shown are untreated cells (UT) and cells infected with non-encapsulated adenovirus (rAd).

FIG. 4: Quantitative PCR results for recombinant adenovirus DNA in livers (black bars), spleens (diagonally hatched bars), kidneys (horizontally hatched bars), and lungs (white bars) of BALB/c mice dosed intravenously with different doses of adenovirus or encapsulated adenovirus. Virus was encapsulated at the highest PEI-mPEG to virus particle ratio (C in Table 1). Groups were injected with 3×10¹⁰, 1×10¹⁰, and 3×10⁹ virus particles per animal. Three animals per group were dosed.

FIG. 5: β-galactosidase activity in livers (black bars), spleens (diagonally hatched bars), kidneys (horizontally hatched bars), and lungs (white bars) of BALB/c mice injected intravenously with different doses of adenovirus or encapsulated adenovirus. Virus was encapsulated at the highest PEI-mPEG to virus particle ratio (C in Table 1). Groups were injected with 3×10¹⁰, 1×10¹⁰, and 3×10⁹ virus particles per animal. Three animals per group were dosed. Due to different background levels in the assay the limit of quantitation was 7.7 ng/g tissue for liver extracts and 0.1 ng/g for all other tissues.

### DETAILED DESCRIPTION OF THE INVENTION

I. Introduction

The present invention relates to a copolymer that noncovalently encapsulates viral particles, forming a complex. The complexes are easily made (e.g. by mixing virus and polymers) and can be used to express nucleic acids in cells. In contrast to covalently attached polymers, chemical modification of a complex biological material such as adenovirus is not required, thereby avoiding extensive characterization of possible reaction products. Introduction of nucleic acids into cells is useful for, e.g., therapeutic or diagnostic purposes (e.g. using a reporter gene). For example, several experimental cancer therapies utilize various aspects of adenovirus or adenovirus vectors. See, for example, U.S. Pat. Nos. 5,846,945; 5,801,029; PCT/US99/08592; U.S. Pat. No. 5,747,469; PCT/US98/03514; and PCT/US97/22036. The virus/polymer complexes of the invention can be used to transfer nucleic acids of interest into different cell types either in vitro, in vivo or ex vivo.

II. The Compositions

A. The Copolymers

In a first aspect, the invention provides a complex comprising an adenovirus noncovalently complexed to a copolymer. The copolymer comprises a structure according to Formula I: 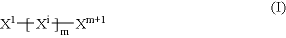
in which m is an integer from 1 to 1,000. The symbol i is an integer from 2 to m and denotes the position of Xⁱ. The symbols X¹, Xⁱ, and X^{m+1} are independently selected monomers, wherein (i) said monomers comprise an amine selected from secondary amines and tertiary amines; and (ii) at least one of said monomers comprise Q. Q is a structure selected from Formula IIa and Formula IIb: 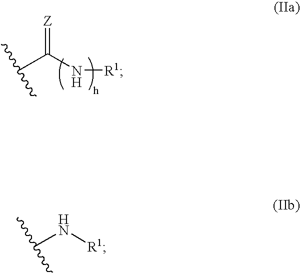
in which Z is selected from the group consisting of O and NH. The symbol h is an integer from 0 to 1. The symbol R¹ comprises a polyalkylene glycol moiety. The copolymers of the invention are also free of cross-polymerization, and, at physiological pH, at least one of the nitrogen atoms in the copolymer is positively charged.

In an exemplary embodiment, at least one of the monomers further comprises a chemical moiety selected from —NH₂ and —OH. If Q is selected from Formula Ia, then at least one Q is covalently linked to said monomers through an atom selected from nitrogen and oxygen. If Q is selected from Formula IIb, then at least one Q is covalently linked to said monomers through a carbon atom.

In an exemplary embodiment, Q is Formula IIa, h is 0, Z is O, and said monomers comprise a structure according to Formula III: 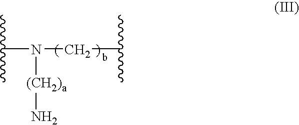
wherein a is an integer from 1 to 10, and b is an integer from 1 to 10. In another exemplary embodiment, Q is Formula IIa, h is 0, Z is O, and said monomers comprise a structure according to Formula IV: 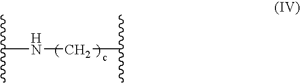
wherein c is an integer from 1 to 10. In yet another exemplary embodiment, Q is Formula IIa, h is 0, Z is O, and said copolymer has: a) at least one monomer with a structure according to Formula III, wherein a is an integer from 1 to 10, and b is an integer from 1 to 10; and b) at least one monomer with a structure according to Formula IV; wherein c is an integer from 1 to 10. In still another exemplary embodiment a is 2. In another exemplary embodiment b is 2. In an exemplary embodiment c is 2. In still another exemplary embodiment a is 3. In another exemplary embodiment b is 3. In an exemplary embodiment c is 3. In another exemplary embodiment, the monomer is ethylene imine.

In an exemplary embodiment, Q is Formula Ia, h is 0, Z is O, and said monomers comprise a structure according to Formula V: 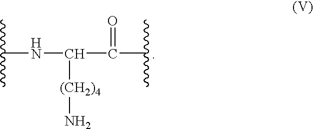
In another exemplary embodiment, the monomer is lysine.

In an exemplary embodiment, Q is Formula Ia, h is 1, Z is NH, and said monomers comprise a structure according to Formula VI: 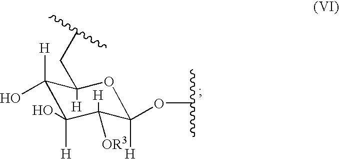
wherein R³ is a member selected from H, —(CH₂CH₂)NH(CH₂CH₃)₂, and —(CH₂CH₂)N(CH₂CH₃)₂CH₂CH₂NH(CH₂CH₃)₂. In another exemplary embodiment, R³ is H for about two of every three of said monomers, and for about one of every three of said monomers, R is a member selected from —(CH₂CH₂)NH(CH₂CH₃)₂, and —(CH₂CH₂)N(CH₂CH₃)₂CH₂CH₂NH(CH₂CH₃)₂. In another exemplary embodiment, the monomer is DEAE-Dextran.

In an exemplary embodiment, Q is Formula IIb, and said copolymer comprises: a) at least one monomer which comprises a structure according to Formula VI, wherein R³ is a member selected from H, —(CH₂CH₂)NH(CH₂CH₃)₂, and -(CH₂CH₂)N(CH₂CH₃)₂CH₂CH₂NH(CH₂CH₃)₂; and b) at least one monomer which comprises a structure according to Formula VII: 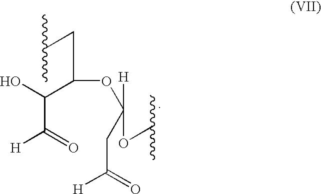
In another exemplary embodiment, R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂. In another exemplary embodiment, the percentage of said monomers which comprise a structure according to Formula VII is between 5 and 25. In still another exemplary embodiment, R³ is H for about two of every three of said monomers, and for about one of every three of said monomers, R is a member selected from —(CH₂CH₂)NH(CH₂CH₃)₂, and —(CH₂CH₂)N(CH₂CH₃)₂CH₂CH₂NH(CH₂CH₃)₂, and the percentage of said monomers which comprise a structure according to Formula VII is between 5 and 25.

In certain embodiments, the non-ionic polymer contains a polyalkylene glycol moiety, which is covalently attached to some of the monomers. In certain embodiments, this covalent attachment results in the formation of a secondary or tertiary amine, an amide, a dihydrazide, an ester, a urea, an isourea, a carbamate, or an urethane. Examples of polyalkylene glycols include polyethylene glycol (PEG) and its derivatives.

In one embodiment, the addition of a non-ionic polymer, such as polyethylene glycol (PEG), to a cationic polymer, such as PEI, polylysine, DEAE-Dextran and variants thereof, prevents precipitation and aggregation of the complexes formed by the copolymer and virus, thus increasing the solubility of the complexes. In another embodiment, PEG functions to increase transfection efficiency.

In another exemplary embodiment, R¹ comprises a structure according to Formula VIII: 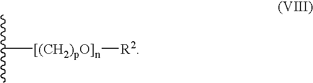
The symbol n is an integer from 2 to 2,000. The symbol p is an integer from 1 to 8. R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl. In yet another exemplary embodiment, p is 2.

The copolymers of the invention have different levels of non-ionic polymer substitution on the monomers of the cationic polymer. For example, 15% of the ethylene imine monomers in the cationic polymer PEI are substituted with the non-ionic polymer PEG. In some cases, the level of non-ionic polymer substitution is between 10% and 20%. In other cases, the level of non-ionic polymer substitution is between 10% and 30%. In other cases, the level of non-ionic polymer substitution is between 15% and 25%. In other cases, the level of non-ionic polymer substitution is about 20%. In other cases, the level of non-ionic polymer substitution is between 10% and 40%.

In an exemplary embodiment, the percentage of monomers which are substituted with Q is at least 10% and Q has a structure according to Formula IX: 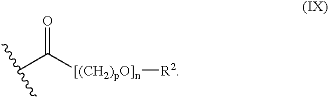
The symbol n is an integer from 2 to 2,000. The symbol p is an integer from 1 to 8. R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl. In yet another exemplary embodiment, p is 2. In another exemplary embodiment, the percentage of monomers which are substituted with Q is from 15 to 30. In still another exemplary embodiment, the percentage of monomers which are substituted with Q is from 17 to 22.

In an exemplary embodiment, the percentage of monomers which are substituted with Q is at least 10 and Q has a structure according to Formula X: 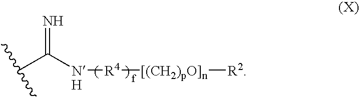
The symbol n is an integer from 2 to 2,000. The symbol p is an integer from 1 to 8. The symbol f is an integer from 0 to 1. The symbol R⁴ has a structure according to Formula XI when f is 1: 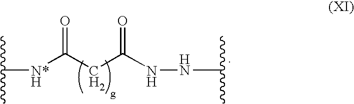
In Figure XI, N′ is covalently attached to N*, and g is an integer from 1 to 9. The symbol R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl. In another exemplary embodiment, f is 0. In yet another exemplary embodiment, f is 1. In still another exemplary embodiment, g is 4. In another exemplary embodiment, the percentage of monomers which are substituted with Q is from 15 to 30. In another exemplary embodiment, the percentage of monomers which are substituted with Q is from 17 to 22.

In an exemplary embodiment, the percentage of monomers which are substituted with Q is at least 10 and Q has a structure according to Formula XII: 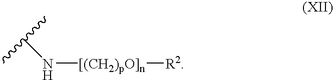
The symbol n is an integer from 2 to 2,000. The symbol p is an integer from 1 to 8. R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl. In another exemplary embodiment, the percentage of monomers which are substituted with Q is from 15 to 30. In another exemplary embodiment, the percentage of monomers which are substituted with Q is from 17 to 22.

In an exemplary embodiment, the complex has a diameter between about 20 nm and about 300 nm. In another exemplary embodiment, the complex has a diameter between about 80 nm and about 150 nm.

In an exemplary embodiment, R² is a member selected from substituted and unsubstituted alkyl, substituted and unsubstituted aryl, folate, transferrin, galactose, glucose, antibodies, antibody fragments, and peptides. In another exemplary embodiment, R² is methyl. In another exemplary embodiment, p is 2. In another exemplary embodiment, p is 2, a is 2, b is 2, and c is 2.

In an exemplary embodiment, the ratio of the copolymer to the adenovirus is between 3,500:1 and 30,000:1. In another exemplary embodiment, the ratio of the copolymer to the adenovirus is between 3,600:1 and 20,000:1. In still another exemplary embodiment, the ratio of the copolymer to the adenovirus is between 10,000:1 and 30,000:1.

The copolymers of the invention can possess a range of physical dimensions. For example, some of the copolymers of the invention have an average molecular weight of between about 100 and about 300 kilodaltons (kDa). In some embodiments, the average molecular weight is between 125 and 250 kDa. In other embodiments, the average molecular weight is between about 150 and about 170 kDa. The length of the copolymer is not critical, as long as the complex it forms with the adenovirus is substantially electroneutral. In an exemplary embodiment, the symbol m is an integer from 10 to 900. In another exemplary embodiment, the symbol m is an integer from 50 to 600. In still another exemplary embodiment, the symbol m is an integer from 75 to 300.

Preparation of the Copolymer

The following exemplary schemes 1-5 illustrate methods of preparing the compounds of the invention. These methods are not limited to producing the compounds listed, but can be used to prepare other compounds as well. The compounds of the invention can also be produced by methods not explicitly illustrated in the schemes. The compounds can be prepared using readily available starting materials or known intermediates.

The copolymers, in their most general form, are formed by reacting a cationic polymer with a non-ionic polymer that, for the exception of one end, is capped with non-reactive groups. For example, the copolymer PEI-mPEG is formed by the reaction of a cationic polymer PEI with a non-ionic polymer PEG which is capped at one end with a methoxy group and possesses a succinimidyl propionate group at the other end.

Non-Ionic Polymers

A nonionic, hydrophilic polymer such as polyalkylene glycol is covalently attached to a cationic polymer in this invention. Suitable polyalkylene glycols are commercially available from many sources, including polypropylene glycol and poly(1,2 butylene glycol) from Aldrich Chemical Company, and polyethylene glycol and its derivatives from Nektar Therapeutics. In some instances, the polyalkylene glycol subunit is covalently attached to the cationic polymer through a group such as a secondary or tertiary amine, an amide, a dihydrazide, an ester, a urea, an isourea, a carbamate, an urethane, or combinations thereof. The number of polyalkylene glycol subunits (n) can be, e.g., between 2-2,000. In some compounds of the invention, the number of subunits is between 45-1,200. In other compounds of the invention, the number of subunits is between 250-1,000.

In order to reduce unwanted side reactions, polyalkylene glycol can be capped, e.g., with a group forming an ether linkage, such as an alkoxy group. In some compounds of the invention, monomethoxypolyethylene glycol (mPEG) is used as a capping group. Other examples of capping groups are substituted and unsubstituted alkyl, substituted and unsubstituted aryl, folate, transferrin, galactose, glucose, antibodies, antibody fragments, and peptides. Still other examples of capping groups for PEG can be found in the Nektar Therapeutics (formerly Shearwater Polymers) (Birmingham, Ala.) 2001 catalog (available on the Internet (World Wide Web) at nektar.com), which is herein incorporated by reference.

In some embodiments, before coupling to the cationic polymer, the non-ionic polymer is chemically activated. Potentially useful forms of, for example, activated PEG (in this example, mPEG) include cyanuric chloride mPEG, succinimidyl succinate mPEG, tresyl-mPEG, and succinimidyl propionate mPEG (mPEG-SPA). Still other examples of activated PEG groups can also be found in the Nektar Therapeutics catalog mentioned above.

Cationic Polymers

The cationic polymers of the invention comprise the symbols X¹, Xⁱ, and X^{m+1} which represent positively charged monomers. These positively charged monomers form a cationic polymer. These positively charged monomers contain secondary or tertiary amines, and can contain primary amine or alcohol functionalities as well. The cationic polymers of the invention can have an average molecular weight of between about 800 and about 800,000 daltons. In some embodiments, the average molecular weight is between 2,000 and 100,000 daltons. In other embodiments, the average molecular weight is between about 15,000 and about 50,000 daltons. Additionally, the length of the cationic polymer, which can be represented by m, can vary between 1 and 1000. In some embodiments, m is a whole number between 10 and 900. In another embodiment, m is a whole number between 50 and 600. In still another embodiment, m is a whole number between 75 and 300.

In an exemplary embodiment, the cationic polymers are polyalkylene imines, polylysine, DEAE-Dextran, and DEAE-Dextran variants.

Cationic Polymers: Polyalkylene Imines

In an exemplary embodiment, the cationic polymer is a polyalkylene imine. In some cases, polyalkylene imine contains the following monomer: 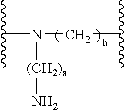
in which, a and b are integers between 1-10. In some cases, a is 2 and b is 2. In other cases, a is 3 and b is 3. In other cases, polyalkylene imine contains the following monomer: 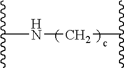
in which, c is an integer between 1-10. In some cases, c is 2. In other cases, c is 3. In still other cases, polyalkylene imine is a mixture of the monomers listed above. In yet another case, polyalkylene imine is polyethylene imine (PEI).

Exemplary polyethylene imine-PEG copolymers are produced by the method of Scheme 1.

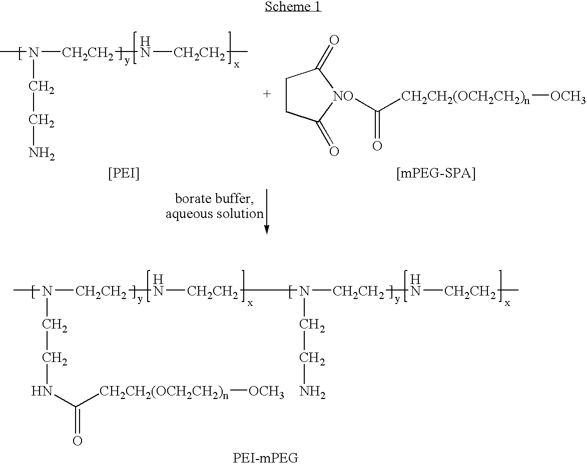

In this scheme, PEI is reacted with a methyl-capped, succinimide-activated PEG in an aqueous solution containing a borate buffer in order to produce a PEI-mPEG copolymer. In certain embodiments polyalkylene imine contains monomers of a structure according to Scheme I, in which the ratio of x to y is between 1:1 and 5:1. The ratio can also be, e.g., 2:1 or even 3:1. Suitable polyalkylene imine compounds are commercially available from many sources, including polyethylenimine from Aldrich Chemical Company, polyethylenimine from Polysciences, and POLYMIN poly(ethylenimine) and LUPASOL™ poly(ethylenimine) available from BASF Corporation.

Cationic Polymers: Polylysine

Another exemplary cationic polymer used in the invention is polylysine. Polylysine is commercially available from many sources, including Sigma Chemical Company. Polylysine is composed of monomers having the following structure:

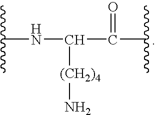

Polylysine-PEG copolymers are produced by the method of Scheme 2.

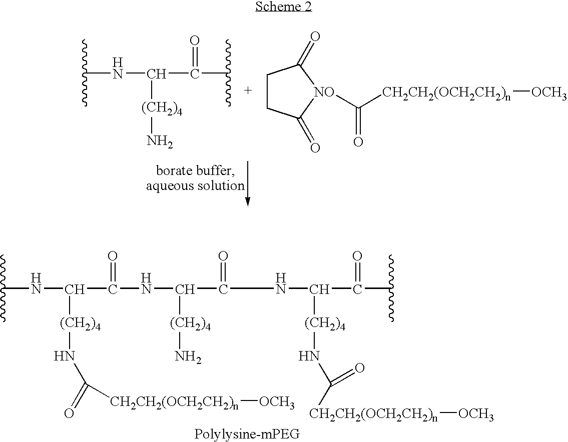

In this scheme, polylysine is reacted with a methoxy-capped, succinimidyl propionate-activated PEG in an aqueous solution containing a borate buffer in order to produce a polylysine-mPEG copolymer.

Cationic Polymers: DEAE-Dextran

Another exemplary cationic polymer used in the invention is DEAE-Dextran. DEAE-Dextran hydrochloride is commercially available from many sources, including Sigma Chemical Company. DEAE-Dextran can contain a mixture of monomers such as the ones listed below:

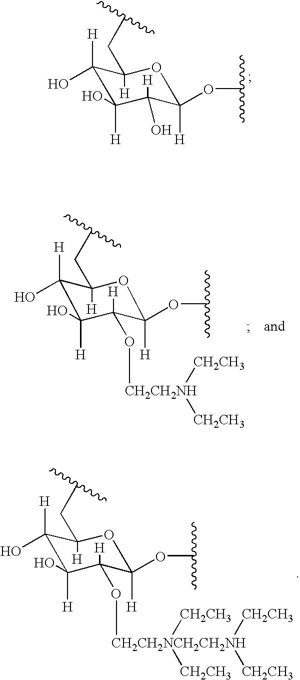

DEAE-Dextran-PEG copolymers are produced by the method of Scheme 3.

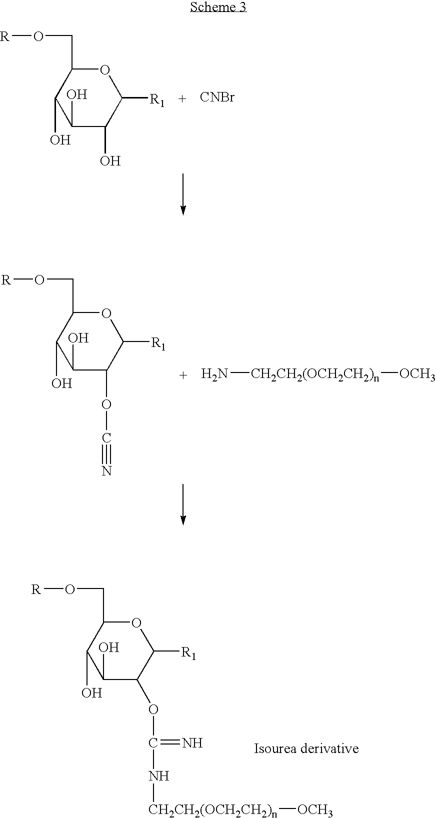

In this scheme, a saccharide is reacted with cyanogen bromide in a 60% acetone:30% water: 10% triethylamine solvent at −15° C. in order to produce an activated saccharide nitrile. When subsequently mixed for 24 hours with an amine-substituted MPEG in aqueous acetate buffer at 4° C., a DEAE-Dextran-mPEG isourea is formed. In those embodiments where DEAE-Dextran is the cationic polymer, the ratio of unsubstituted glucose subunits to DEAE-substituted glucose (including single and multiple DEAE-substitutions) subunits is between 1:1 and 5:1. The ratio can also be, e.g., 2:1 or even 3:1.

In another exemplary embodiment, DEAE-Dextran-PEG copolymers with an adipate dihydrazide linker are produced by the method of Scheme 4.

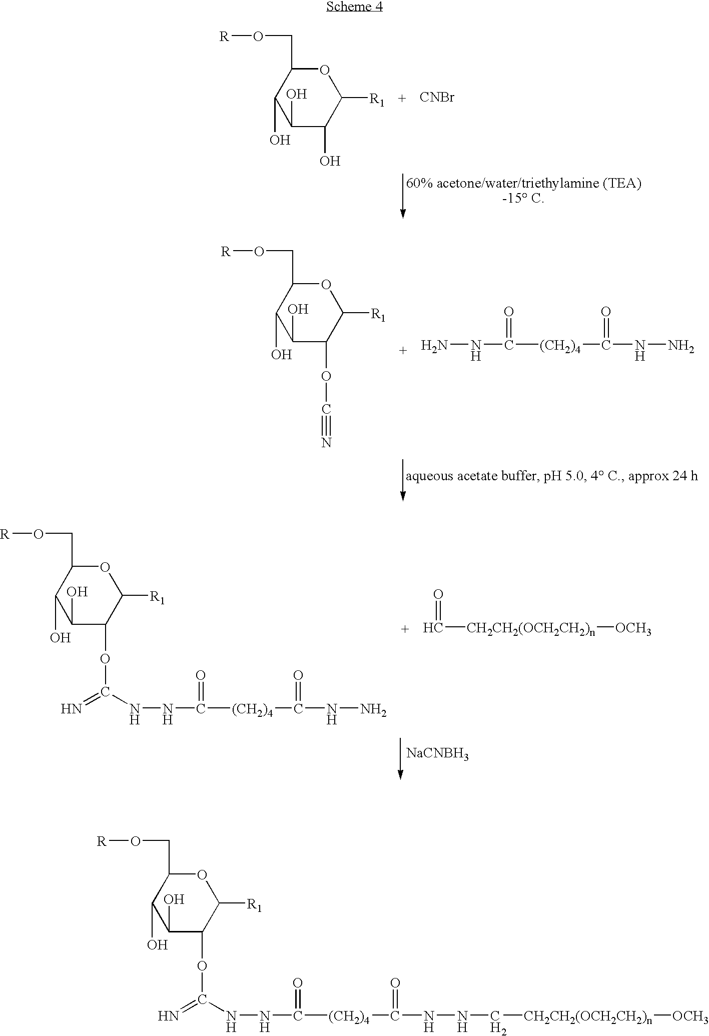

In this scheme, a saccharide is reacted with cyanogen bromide in a 60% acetone:30% water: 10% triethylamine solvent at −15° C. in order to produce an activated saccharide nitrile. The activated polymer is first reacted with adipic acid dihydrazide, and subsequently reacted with an aldehyde functionalized mPEG in the presence of sodium cyanoborohydride to furnish DEAE-Dextran-PEG copolymers with an adipate dihydrazide linker. In those embodiments where DEAE-Dextran is the cationic polymer, the ratio of unsubstituted glucose subunits to DEAE-substituted glucose (including single and multiple DEAE-substitutions) subunits is between 1:1 and 5:1. The ratio can also be, e.g., 2:1 or even 3:1.

Cationic Polymers: DEAE-Dextran variant

In another exemplary embodiment, certain DEAE-Dextran-PEG copolymer variants are produced. DEAE-Dextran variants can contain the monomers listed above. DEAE-Dextran-PEG copolymer variants are produced by the method of Scheme 5.

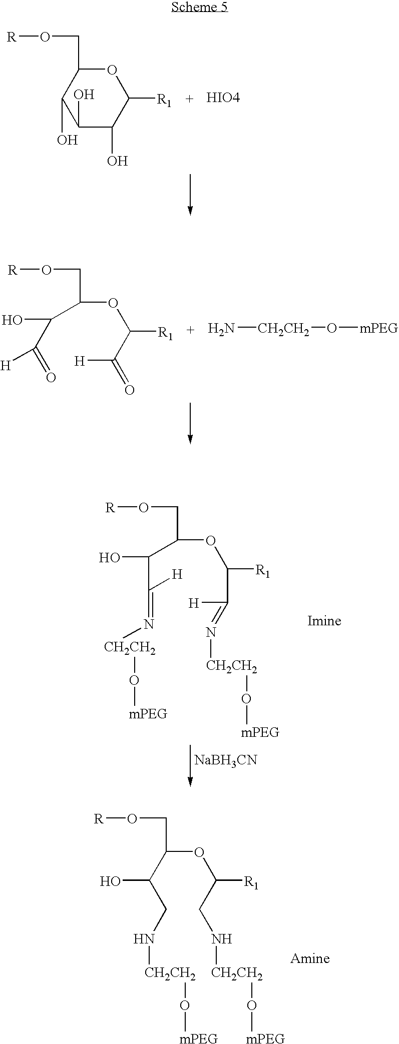

In this scheme, a saccharide is reacted with periodic acid in order to oxidize viccinal hydroxide groups to dialdehydes. When subsequently mixed with an amine-substituted mPEG, a DEAE-Dextran-mPEG imine copolymer variant is formed. Subsequent reduction with sodium cyanoborohydride produces a DEAE-Dextran-mPEG amine copolymer variant.

B. The Virus

Viruses are highly efficient at nucleic acid delivery to specific cell types, while often avoiding detection by the infected hosts' immune system. These features make certain viruses attractive candidates as gene-delivery vehicles for use in gene therapies. Retrovirus, adenovirus, adeno-associated virus (AAV), and herpes simplex virus are examples of commonly used viruses in gene therapies. Each of the aforementioned viruses has its advantages and limitations, and can therefore be selected according to its suitability for a given gene therapy.

Adenoviruses as vectors for the delivery of foreign DNA are used extensively as tools of modern molecular biology. Adenoviral replication does not require the recipient host cell to be dividing, in contrast to most retrovirus vectors. Adenoviruses can be designed to enter mammalian cells and express proteins but can be otherwise defective for production of infectious progeny virus.

Adenovirus vectors can be prepared to be either replication competent or conditionally defective in a variety of genes required for a productive infection. For instance, from experience with adenovirus-SV40 recombinants, it was learned that the entire adenovirus E3 region could be deleted without a major change in viral growth in tissue-cultured cells. This region can be substituted with foreign DNA. The resulting adenovirus can be grown in any cell line permissive for wild-type adenovirus infection. E3-substituted adenoviruses have been used primarily for the insertion of genes to produce proteins for immunization.

In contrast to adenoviruses constructed for use as vectors for immunization where viral replication is desired to increase the amount of the immunizing epitopes, viruses engineered as tools for gene therapy are generally designed to be defective for replication. Most of the foreign DNA for the latter vectors replace the deleted E1A and E1B regions. The constructs are made by a variety of protocols but include plasmids into which the foreign DNA is inserted, flanked by adenovirus sequences.

There are other ways in which adenoviruses can be used to facilitate the insertion of foreign DNAs into cells. The mechanisms of viral attachment, processing through the endosomes, and eventual delivery of DNA to the nucleus can be used to co-internalize foreign DNA external to the adenovirus particle. For example, DNA complexed with polylysine can be joined to adenovirus particles and co-internalized. See, e.g., Cottam, J Virol 67: 3777-3785 (1993), Wagner, PNAS 88: 4255-5259 (1991). The complex enters cells presumably by attachment of the fiber to its putative receptor, but there have been modifications of these techniques to include ligands, like transferrin, to facilitate entry into cells with the transferrin receptor. See, e.g., Wagner, PNAS 89: 6099-6103 (1992). It is unnecessary to link the virus and the external DNA for efficient entry of the DNA into cells in culture. See, e.g., Yoshimura, J Biol Chem 268: 2300-2303 (1993).

C. The Complex

In another aspect, the invention provides complexes comprising an adenovirus and a copolymer. These copolymers can be of a kind described in part A above. In these complexes, the adenovirus and copolymer are noncovalently complexed to one another.

The complex of the invention can possess a range of physical dimensions. For example, the amount of the copolymer versus the amount of adenovirus in the complex can vary. In some embodiments, the ratio of the copolymer to the adenovirus is between 3,500:1 and 30,000:1. In other embodiments, the ratio of the copolymer to the adenovirus is between 3,600:1 and 20,000:1. In still other embodiments, the ratio of the copolymer to the adenovirus is between 10,000:1 and 30,000:1.

Additionally, the size of the complex can vary. In some embodiments, the complex is between about 20 nm and about 300 nm. In other embodiments, the complex is between about 80 nm and about 150 nm.

Preparation of the Complex

In its simplest form, the copolymers of the invention are simply contacted to, or mixed with, an adenovirus (See Example 2). Complex formation occurs almost instantaneously, and no further purification is needed. The complexes of the invention can then be used for contacting a cell. Contacting the complexes of the invention to cells results in the introduction of the virus or viral nucleic acids into the cell.

Non-covalent encapsulation of adenovirus with cationic PEI-PEG copolymer results in a complete masking of virus surface charges while increasing virus infection and transgene expression in vitro and in vivo. This surface modification was achieved through a simple mixing process, with no further processing steps. PEI-PEG can be added to any purified virus preparation as a formulant, thereby allowing its use with any previously generated viral vectors.

In some embodiments, the cells are contacted with the complex in vitro. In some embodiments, the cells are contacted with the complex in vivo.

In another aspect, the invention provides a method of preparing a noncovalently complexed adenovirus copolymer complex. In this method, the copolymer, as described in this section, is contacted with an adenovirus.

In another aspect, the invention provides a method of introducing an adenovirus into a cell. In this method, a copolymer is noncovalently contacted to an adenovirus, which forms the complex. This complex is then contacted to a cell.

IV. Pharmaceutical Applications

In a fourth aspect, the invention provides a physiological formulation comprising: (a) a copolymer; (b) an adenovirus, which forms a noncovalent complex with the adenovirus; and (c) a physiologically acceptable excipient. The copolymer in this physiological formulation comprises a structure according to Formula I wherein m is an integer from 1 to 1,000. The symbol i is an integer from 2 to m and denotes the position of Xⁱ. The symbols X¹, Xⁱ, and X^{m+1} are independently selected monomers, wherein (i) the monomers comprise an amine selected from secondary amines and tertiary amines; and (ii) at least one of the monomers comprise Q. Q is a structure selected from Formula Ia and Formula IIb, wherein Z is selected from the group consisting of O and NH. The symbol h is an integer from 0 to 1. The symbol R¹ comprises a polyalkylene glycol moiety. The copolymers of the invention are also free of cross-polymerization, and at physiological pH, at least one of the nitrogen atoms in the copolymer is positively charged.

Physiologically acceptable excipients include, e.g., materials such as carriers, water, pH-adjusting or buffering agents, preservatives, stabilizers or other ingredients. A physiologically acceptable carrier can contain a physiologically acceptable compound that acts, for example, to stabilize the recombinant adenoviral vector delivery system. A physiologically acceptable compound can include, for example, carbohydrates, such as glucose, sucrose or dextrans, hydroxypropyl-β-cyclodextrin, antioxidants, such as ascorbic acid or glutathione, chelating agents, water, low molecular weight proteins or other stabilizers or excipients.

Other physiologically acceptable compounds include, for example, wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of physiologically acceptable carrier depends on the route of administration and the particular physiochemical characteristics of the recombinant adenoviral vector delivery system. Examples of carriers, stabilizers or adjuvants can be found in Gennaro, REMINGTON'S: THE SCIENCE AND PRACTICE OF PHARMACY, 19th ed. (1995) Mack Publishing Co., Easton, Pa., incorporated herein by reference.

The compounds of the present invention can be prepared and administered in a wide variety of oral, parenteral and topical dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. Accordingly, the present invention also provides physiological compositions comprising a physiologically acceptable excipient and a complex composed of a copolymer from the “Copolymer” section provided above, and a virus from the “Virus” section provided above.

An effective amount of the complex is administered to a patient as a composition in a pharmaceutically acceptable excipient, including, but not limited to, saline solutions, suitable buffers, preservatives, stabilizers, and may be administered in conjunction with suitable agents such as antiemetics. An effective amount is an amount sufficient to effect beneficial or desired results, including clinical results. An effective amount can be administered in one or more administrations. For purposes of this invention, an effective amount of a complex is an amount that is sufficient to palliate, ameliorate, stabilize, reverse, slow or delay the progression of the disease state or to diagnose a particular tissue or disease state. Some individuals are refractory to these treatments, and it is understood that the methods encompass administration to these individuals. The amount to be given will be determined by the condition of the individual, the extent of disease, the route of administration, how many doses will be administered, and the desired objective.

Delivery of the complex is generally accomplished by either site-specific injection or intravenous injection. Site-specific injections of vector may include, for example, intraperitoneal, intrapleural, intrathecal, intra-arterial, intra-ocular, intra-tumor injections or topical application. These methods are easily accommodated in treatments using the combination of the complex and other agents, as appropriate.

The complexes may be delivered to the target cell in a variety of ways, including, but not limited to, liposomes, general transfection methods that are well known in the art (such as calcium phosphate precipitation or electroporation), direct injection, and intravenous infusion. The means of delivery will depend in large part on the particular complex (including its form) as well as the type and location of the target cells (i.e., whether the cells are in vitro or in vivo). Depending on the route of administration, the viral dosage amounts for humans can range from 1×10⁶ and 1×10¹⁴.

Liquid form preparations include solutions, suspensions, and emulsions, for example, water or water/propylene glycol solutions. For parenteral injection, liquid preparations can be formulated in solution in aqueous polyethylene glycol solution.

V. Kits

In a fifth aspect, the invention provides a kit comprising a copolymer and an adenovirus, wherein the copolymer and adenovirus are noncovalently attached. The copolymer of the kit comprises a structure according to Formula I wherein m is an integer from 1 to 1,000. The symbol i is an integer from 2 to m and denotes the position of Xⁱ. The symbols X¹, Xⁱ, and X^{m+1} are independently selected monomers, wherein (i) said monomers comprise an amine selected from secondary amines and tertiary amines; and (ii) at least one of said monomers comprise Q. Q is a structure selected from Formula IIa and Formula IIb, wherein Z is selected from the group consisting of O and NH. The symbol h is an integer from 0 to 1. The symbol R¹ comprises a polyalkylene glycol moiety. The copolymers of the invention are also free of cross-polymerization, and at physiological pH, at least one of the nitrogen atoms in said copolymer is positively charged.

This invention also provides kits for the preparation of viruses for the infection of cells, e.g., for gene therapy. The kits comprise, for example, the copolymers of the invention. Optionally the kits can contain a virus such as an adenovirus in a separate container. The kit can optionally contain written instructions describing how to use the invention. Other materials useful in the performance of the assays can also be included in the kits, including test tubes, transfer pipettes, and the like. The kits of the present invention can contain materials sufficient for one assay, or can contain sufficient materials for multiple assays.

The following examples are offered by way of illustration and not by way of limitation.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

General

In the examples below, unless otherwise stated, temperatures are given in degrees Celsius (° C.); operations were carried out at room or ambient temperature, “rt,” or “RT,” (typically a range of from about 18-25° C.); evaporation of solvent was carried out using a rotary evaporator under reduced pressure (typically, 4.5-30 mm Hg) with a bath temperature of up to 60° C.; the course of reactions was typically followed by TLC and reaction times are provided for illustration only; yields are provided for illustration only; and the following conventional abbreviations are also used: L (liter(s)), mL (milliliters), mmol (millimoles), g (grams), mg (milligrams), min (minutes), and h (hours).

Unless otherwise stated, all chemicals used in the synthesis were purchased from Sigma (St. Louis, Mo.). ¹H-NMR analysis was conducted by Numega Resonance Labs, San Diego, Calif. Prior to NMR analysis, the copolymer was dialyzed against deionized water using a 10,000 MWCO membrane (Slide-a-Lizer, Pierce, Rockford, Ill.) and lyophilized. Recombinant adenovirus expressing a LacZ fusion protein (β-Gal) gene under the control of the CMV immediate early promoter (BGCG) was previously described (Wills, K. N., et al. Hum Gene Ther. 5:1079-1088 (1994)). Recombinant adenovirus encoding the green fluorescent protein (GFP) gene under the control of the same CMV immediate early promoter (GFCB) was previously described (Rahman, A., et al., Mol Ther. 3:768-778 (2001)). BGCG and GFCB were produced and purified as described (Huyghe, B. G., et al., Hum Gene Ther. 6:1403-1416 (1995)).

Size exclusion chromatography was conducted on a Akta FPLC system using a HiPrep column (1.6×60 cm) packed with Sephacryl S-200 resin (Amersham Biosciences, Piscataway, N.J.). Reverse phase chromatography was conducted on a Jupiter 5u C4 column (150×2 mm, Phenomenex Torrance, Calif.) using a Waters HPLC system (Waters, Milford, Mass.) and a Sedex 75 evaporative light scattering detector (SEDERE, Alfortville, France). Complex particle size was determined by dynamic light scattering on a N4 Plus particle sizer (Coulter, Miami, Fla.). Surface charge (zeta potential) was measured on a DELSA 440 SX (Coulter, Miami, Fla.). Coated viral particles were analyzed by anion-exchange chromatography on a Resource Q column (Amersham Biosciences, Piscataway, N.J.) (Shabram, P. W., et al. Hum Gene Ther. 8:453-465 (1997)). Flow cytometry was conducted on a FACS Calibur flow cytometer (Becton Dickinson, San Jose, Calif.); forward scatter (FSC), side scatter (SSC) and FL-1 parameters were collected for a total of 50,000 cells. Total protein content of cell and tissue lysates was determined by the BCA method (Pierce, Rockford, Ill.) relative to a bovine serum albumin (BSA) standard.

### Example 1

Preparation of PEI-mPEG Polymer

PEI (25 kDa) was purchased from Aldrich (Milwaukee, Wis.), and methoxy-PEG-SPA (5 kDa) was obtained from Shearwater (Huntsville, Ala.). Analysis of the starting materials by reverse phase HPLC (linear gradient from 100% of 0.1% TFA in water to 100% of 0.1% TFA in acetonitrile, 20 minutes, flow rate: 0.4 mL per minute) showed a single peak at 10 minutes for PEI and 15 minutes for MPEG.

PEG grafted polymer was synthesized by reaction of PEI with mPEG-SPA as shown in Scheme 1. In Scheme 1, a 1M PEI solution was prepared by dissolving 2.155 grams of PEI (25 kDa) in 10 mL of water, adjusting the pH to 7.0 with 1N HCl, and adjusting the volume to 50 mL with water. To 153.9 milligrams of mPEG-SPA (3.0×10⁻⁵ mol) was added 400 μL of PEI stock solution (1.27×10⁴ mol primary amines) diluted in 800 μL of borate buffer (200 mM borate, 150 mM NaCl, 1 mM EDTA, pH 8.4). The reaction was allowed to progress for 1 hour at room temperature with shaking.

Purification of PEI-mPEG Polymer

The reaction mixture was purified by size exclusion chromatography. Fractions were collected; those containing PEI-mPEG copolymer were pooled and the pool analyzed by reverse phase chromatography.

Characterization of PEI-mPEG Polymer

Analysis of the purified reaction product (PEI-mPEG copolymer) by reverse phase chromatography showed a single broad peak. The lack of the characteristic PEI and PEG peaks demonstrated the removal of the starting materials. Additionally, a broad PEI-mPEG elution peak suggested a heterogeneous distribution of PEG to PEI ratios in the copolymer.

The ratio of PEG to PEI in the copolymer was determined by comparing the ¹H-NMR peak areas of the —CH₂—CH₂—O— protons in PEG and the —CH₂CH₂N— protons of PEI. Based on this data, the PEI-MPEG copolymer contained an average of 27.2 PEG chains per PEI molecule. This corresponds to a degree of modification of 18.8% of primary amines and an average molecular weight of 161 kDa.

### Example 2

Preparation of the Complex

Recombinant adenovirus was treated with PEI-mPEG copolymer at various ratios by (1) diluting adenovirus into phosphate buffered saline (PBS) or vPBS (1×PBS, 3% w/v sucrose, 2 mM magnesium chloride, pH 7.5) to a final concentration of 1×10¹¹ particles per mL, (2) adding PEI-mPEG stock, and (3) mixing by pipetting. Complex formation was allowed to occur for 15 minutes at room temperature before use. No further purification was needed.

Characterization of the Complex

Characterization of the adenovirus before and after encapsulation was analyzed by the following methods: Dynamic light scattering, zeta-potential determination, anion-exchange chromatography, and isopycnic CsCl density gradient centrifugation. PEI-MPEG encapsulated adenovirus was diluted 10-fold in ultrapure water prior to analysis. The complexes of the invention were physically characterized as shown in Table 1.

TABLE 1

| | μg/mL | Particle size | Zeta-potential |
|---|---|---|---|
| Formulation | PEI-mPEG | [nm] | [mV] |
| rAd | 0 | 123.0 | −29.2 |
| rAd-PEI-mPEG A | 20 | 164.5 | −10.2 |
| rAd-PEI-mPEG B | 99 | 130.7 | 0 |
| rAd-PEI-mPEG C | 494 | 129.6 | 0 |

Particle size was determined by dynamic light scattering. The reported hydrodynamic diameter was determined by measuring the Brownian motion of adenovirus particles in an aqueous buffer. Addition of PEI-mPEG copolymer resulted in a reduction of the surface charge in a dose-dependent manner and approached neutrality at the higher polymer concentrations. Since the surface charges were masked, it was surmised that the adenovirus particle was encapsulated with PEI-mPEG polymer. This binding occurred through charge interaction between the polycationic PEI backbone and the negatively charged virus surface.

Analysis of the particle size by dynamic light scattering showed only a slight increase at the higher polymer concentrations. At the lowest concentration, however, a larger increase in average particle size was observed. The latter may be due to polymer mediated particle aggregation, which is more likely to occur for polymer concentrations at which the virus particle surface is only partially saturated with polymer.

In order to determine if the encapsulated adenovirus was stable in the presence of serum, PEI-MPEG treated adenovirus was incubated for one hour in 50% v/v fetal bovine serum (FBS) in PBS at room temperature. The sample was subsequently tested by Resource Q HPLC analysis. No adenovirus peak was observed at the “typical” adenovirus retention time, indicating that the polymer was not displaced from the rAd by FBS. The retention time of untreated control adenovirus incubated in 50% v/v FBS did not change.

An aliquot of the PEI-MPEG treated virus was subjected to isopycnic CsCl density gradient centrifugation. Column purified BGCG and BGCG in PEI-mPEG were each loaded onto CsCl step-gradients as described previously (Prage, L. et al., Virology. 49:745-757 (1972)) with minor modifications. Approximately 5×10¹¹ virus particles from each preparation were overlaid on 1.25 gm/ml and 1.40 gm/ml CsCl step-gradients in 10 mM Tris-HCl buffer, pH 8.0. The virus samples were centrifuged in a Beckman SW41 Ti rotor for 1 hour at ˜154,000×g, 8° C. The virus band from each tube was collected and mixed with 1.30 gm/ml CsCl in 10 mM Tris-HCl, pH 8.0. Centrifugation was continued overnight in a VTi 65.3 rotor at ˜199,000×g, 8° C. Each virus band was collected, dialyzed in 3% sucrose in 2 mM MgCl and 1×PBS, pH 7.4 using Spectra/Por membrane with MWCO 50,000 (Spectrum Medical Industries, Inc., Houston, Tex.). Dialyzed virus was stored frozen at −80° C.

Only one band with the same buoyant density as control adenovirus was seen. The isolated adenovirus band was dialyzed against vPBS (1×PBS, 3% sucrose, 2 mM magnesium chloride, pH 7.5) and analyzed by Resource-Q HPLC. Using standard chromatographic conditions, the virus eluted at the typical retention time, suggesting that the PEI-mPEG polymer was bound to the virus by charge interaction and was displaced by the high ionic strength of the CsCl solution. Furthermore, electron microscopy of PEI-mPEG coated virus showed a typical, icosahedral adenovirus morphology. This data shows that the PEI-MPEG polymer interaction with adenovirus was reversible and did not affect the integrity of the virus particle.

### Example 3

In Vitro Infectivity Assay

The effect of encapsulating adenovirus with PEI-mPEG polymer on infectivity and transgene expression was evaluated in vitro on two different cell lines, A549 and T24. A549 cells, human epithelial lung carcinoma cells, express the Coxackie Adenovirus Receptor (CAR) and therefore are easy to infect with adenovirus. A549 cells were maintained in DMEM supplemented with 10% FBS at 37° C. in a 7% CO₂ incubator. In contrast, T24 cells are CAR negative and much less infectible than A549 cells. T24, human epithelial bladder carcinoma cells, were maintained in a 1:1 mixture of Ham's F12/DME high glucose supplemented with 10% FBS and propagated in the same incubator. Cultures were grown in T-225 cm² tissue culture flasks until approximately 80% confluent, detached using 0.25% trypsin and seeded at 5×10⁵ cells per well in 6-well plates. Cells were maintained overnight (37° C., 7% CO₂) before infection with adenovirus or PEI-mPEG encapsulated adenovirus. Infectivity was determined using a recombinant adenovirus expressing the green fluorescent protein (GFP). The transduction frequency, i.e. percentage of GFP positive (infected) cells, was determined by flow cytometry. Transduction frequency (percent GFP positive cells) was determined by dividing the number of FITC (FL-1) positive cells by the total number of analyzed cells.

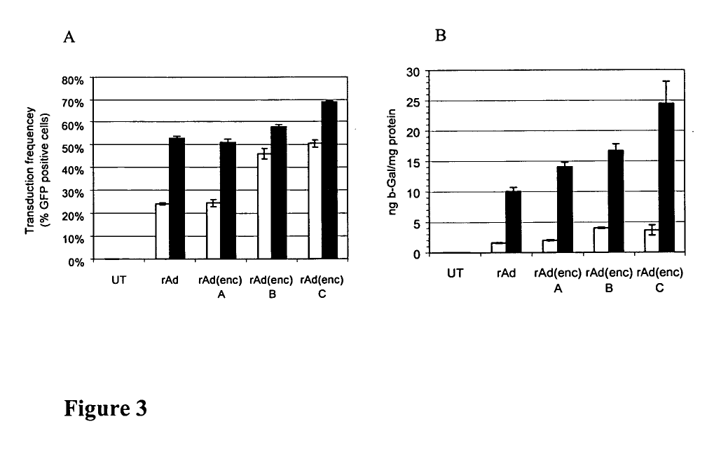

For both cell lines, increases in transduction frequency were directly proportional to an increasing PEI-MPEG concentration (FIG. 3A). For T24 cells, increases relative to non-encapsulated adenovirus were 1%, 89% and 107% for rAd-PEI-mPEG A, B and C, respectively. For A549 cells, a decrease of −3% was observed for encapsulation level A, and increases of 10% and 31% were observed for encapsulation levels B and C, respectively.

Infectivity of encapsulated adenovirus in vitro was increased regardless of the CAR expression status of the cell lines used. While increases over base level (established with non-encapsulated adenovirus) were slightly higher for CAR negative cells (T24), highest transfection levels were achieved with CAR positive cells (A549). This shows that virus encapsulation did not ablate receptor-mediated infection but rather enhanced infectivity in a receptor-independent manner. This observation is in contrast to results achieved with covalent pegylation of adenovirus (O'Riordan, et al. Hum Gene Ther. 10: 1349-1358 (1999); Croyle et al., Hum. Gene Ther. 11:1713-1722 (2000)), where a moderate to severe decrease in in vitro infectivity was observed, depending on the pegylation chemistry used.

Increases in infectivity in vitro have been reported with adenovirus coated with PEI (no PEG) and other polycations (McKay et al., Gene Ther. 7: 644-652 (2000)). Particles larger than 200 nm have been shown to accumulate in the spleen and are therefore efficiently removed from the circulation (Litzinger et al., Biochim. Biophys. Acta. 1190:99-107 (1994)). Additionally, extravasation into certain target tissues such as solid tumors requires particle sizes of less than 300 nm (Hobbs et al., Proc. Natl. Acad. Sci. USA. 95:4607-4612 (1998)). Formulation with PEI-PEG copolymer on the other hand caused no adenovirus aggregation.

In Vitro Transgene Expression

Transgene expression in vitro was evaluated in the same two cell lines using recombinant adenovirus expressing β-galactosidase (FIG. 3B). Cells were infected with BGCG or BGCG encapsulated with PEI-MPEG in the same manner as described for the infectivity assay. Cells were lysed and transgene expression analyzed using a chemiluminescent β-galactosidase reporter gene assay kit (Roche, Mannheim, Germany). Briefly, 24 h post infection cells were washed with PBS and lysed with a mild detergent for 30 minutes at room temperature. After removing cellular debris by centrifugation, 50 μl of diluted cell extract was transferred to a 96-well chemiluminescence assay plate. To this was added 100 μl of chemiluminescence substrate (Galacton Plus) followed by an incubation for 1 hour at room temperature. The assay plate was then analyzed on a TR717 microplate luminometer (Tropix, Bedford, Mass.). β-galactosidase concentration was determined relative to a β-galactosidase standard (provided with kit) and normalized with the total protein content of the lysates. For both cell lines an increase in transgene expression was seen for PEI-MPEG encapsulated adenovirus over non-encapsulated adenovirus. Increases were 27% (A), 157% (B), and 130% (C) for T24 cells and 40% (A), 67% (B), and 144% (C) for A549 cells.

### Example 4

Biodistribution and Transgene Expression In Vivo

Biodistribution and transgene expression was evaluated after intravenous injection of the recombinant adenovirus or encapsulated adenovirus into the tail veins of BALB/c mice. Organs/tissues were harvested three days post injection and analyzed for the presence of adenoviral DNA by quantitative PCR and for β-galactosidase enzyme activity using a chemiluminescent 1-gal reporter gene assay kit.

Biodistribution and Transgene Expression In Vivo

Female BALB/c mice were slowly (300 μl in 20 seconds) injected with BGCG or BGCG encapsulated with PEI-mPEG into the tail vein. Three days after dosing, animals were sacrificed and the livers and tissues resected. The tissues were immediately placed in OCT and snapped frozen at −70° C. 6 μm sections were cut and then stained with X-gal. Sections of each tissue were snap frozen in liquid nitrogen. There were 3 animals per dosing group.

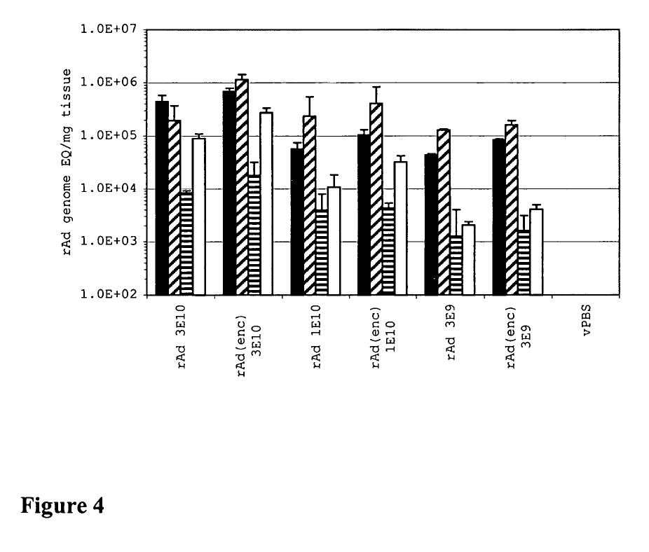

The highest concentration of adenovirus DNA was found in the liver and spleen, whereas the lowest amounts were observed in the kidney (FIG. 4). This pattern remained essentially the same for adenovirus encapsulated with PEI-MPEG. Adenovirus DNA concentration increased in all the organs in a dose-dependent manner for both non-encapsulated and encapsulated adenovirus. Comparing viral DNA concentrations for animals dosed with the same particle number of non-encapsulated and encapsulated adenovirus, an increase in adenoviral DNA copy number per mg of tissue was seen in groups that received the encapsulated adenovirus. Average increases were 1.8-fold for liver, 2.9-fold for spleen, 1.5-fold for kidney, and 2.7-fold for lung.

Transgene Expression In Vivo

β-galactosidase enzyme activity in tissue samples: Tissue samples were weighed into lysing matrix tubes (Q Biogene, Carlsbad, Calif.) and 0.2 mL of detergent lysis buffer with protease inhibitor cocktail (provided with β-Gal kit) per 100 mg of tissue was added. Tissue samples were lysed in a FastPrep tissue homogenizer (Q Biogene, Carlsbad, Calif.). After undergoing 3 freeze-thaw cycles, lysates were incubated at 50° C. for 1 hour to inactivate endogenous β-galactosidase activity. Lysates were clarified by centrifugation at 14,000×g for 10 minutes. Supernatant was removed and diluted aliquots analyzed for β-Gal enzyme activity using a chemiluminescent β-gal reporter assay kit (Roche, Mannheim, Germany).

Assessment of viral DNA levels: DNA was extracted from approximately 100 mg of tissue using Tri-Reagent (Molecular Research Center, Inc.) per the manufacturer's protocol. Quantification of viral DNA was performed using real time quantitative PCR which uses the 5′ nuclease activity of Taq polymerase to detect PCR amplicons (Wen, S. F., et al. Cancer Gene Ther. 7:1469-1480 (2000)). In addition to BGCG, relative quantification of murine GAPDH DNA was assessed to ensure the consistency and quality of DNA.

Primer and probe sequences used for PCR were as follows: BGCG forward primer, 5′-AACGGTACTCCGCCACC-3′; BGCG reverse, 5′-ACTGGTTAGACGCCTTTCTCGA-3′; and BGCG probe, FAM-TCCGCATCGACCGGATCGG-TAMRA; murine GAPDH forward, 5′GAAGGTGAAGGTCGAGTC-3′, and GABDH reverse 5′-GAAGATGGTGATGGGATTTC-3′. The probe was FAM-CAAGCTTCCCGTTCTCAGCC-TAMRA. The PCR thermal profile was 50° C. for 2 minutes, 95° C. for 10 minutes, and 40 cycles of 95° C. for 15 seconds and 62° C. for 1 minute. For viral DNA quantification, diluted viral DNA isolated from BGCG was used as a viral DNA standard. Data from Q-PCR was expressed as viral copies/mg of tissue. The detection limit was approximately 10 copies/mg of tissue.

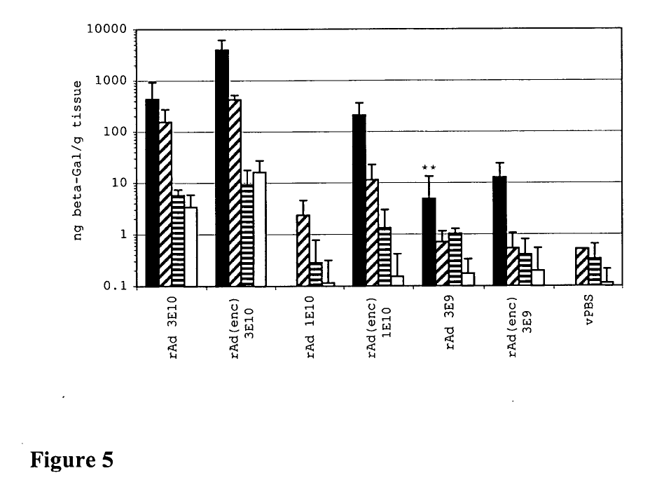

Transgene expression was analyzed by measuring β-galactosidase enzyme activity in extracts prepared from aliquots of the harvested tissues (FIG. 5). For non-encapsulated adenovirus, β-gal activity was at background levels for all doses except at 3×10¹⁰ particles. One exception was the liver sample from one animal dosed at 3×10⁹ particles, which showed a doubling of background expression (14.6 ng/g tissue). In contrast, animals dosed with PEI-mPEG encapsulated adenovirus showed dose-dependent expression throughout the dosing range. As with non-encapsulated adenovirus, expression was highest in liver, followed by spleen, and lowest in kidneys and lung. An increase in expression was observed in all tissues of animals dosed with the encapsulated virus when compared to groups that received the same dose of non-encapsulated adenovirus. The largest increases were seen in liver tissues of animals, i.e., 9.1-fold for animals dosed with 3×10¹⁰ particles, greater than 27.2-fold for animals dosed with ×10¹⁰ particles (based on LOQ), and 2.6-fold for animals dosed with 3×10⁹ particles.

Transgene expression in the liver was visualized by histological analysis of tissue cryo-sections. This analysis was used as a means to assess transduction frequency in vivo. Some mice were injected with various doses of BGCG while others were injected with various doses of PEI-mPEG-BGCG. While the number of transgene expressing cells was approximately equal for animals dosed at 3×10¹⁰ particles, increased expression was observed for animals treated with PEI-MPEG encapsulated virus at the other dose levels. Very few positive cells were detected in the livers of animals dosed with non-encapsulated adenovirus, while a significant number of positive cells were detected when PEI-mPEG encapsulated rAd was injected.

Intravenous injection of recombinant adenovirus results in a highly non-linear dose response in various immune competent mouse strains. This was observed in various organs but mostly reported in the liver, the organ with the highest expression after i.v. injection (Ziegler et al., Hum Gene Ther. 13: 935-945 (2002), Tao et al., Mol Ther. 3: 28-35 (2001)). This effect is attributed to the existence of a “biological filter” which manifests itself by preferential uptake of adenovirus by Kupffer cells as well as other parts of the reticuloendothelial system (Tao et al., Mol Ther. 3:28-35 (2001)). Depletion of Kupffer cells resulted in an increased expression in hepatocytes, supporting the hypothesis that adenovirus is sequestered and degraded in these cells (Ziegler et al., Hum Gene Ther. 13: 935-945 (2002), Tao et al., Mol Ther. 3:28-35 (2001)). Moreover, coadministration of an unrelated adenovirus resulted in increased transgene expression, suggesting that the RES system has a limited capacity and can be saturated by high doses of virus (Ziegler et al., Hum Gene Ther. 13:935-945 (2002)).

Encapsulation of adenovirus with PEI-PEG copolymer provides a practical strategy to reduce the interaction of recombinant adenovirus with the RES and improve the therapeutic potential of the vector system. At 72 hours post administration, viral DNA levels were higher in all tissues for encapsulated adenovirus when compared to non-encapsulated virus injected intravenously at the same dose. This suggests encapsulation with PEI-PEG protected the adenovirus from clearance. At the same time, expression in all analyzed tissues was increased with the largest increases seen in the liver. These findings are consistent with a mechanism of action in which PEI-PEG encapsulation reduces the uptake and degradation of recombinant adenovirus by the RES system, therefore making the rAd more accessible to target tissues resulting in both higher viral DNA and higher expression levels. Moreover, PEI-PEG encapsulation increases the frequency of transduction events in the liver. This effect is more pronounced at low to intermediate viral doses, where the RES system is not saturated with adenoviral particles. This is a valuable finding since increases in dose to achieve higher expression are often prohibited by dose-dependent toxicity. Taken together, these results show that adenovirus encapsulation with PEI-PEG polymer provides an interesting strategy to improve the therapeutic effectiveness of adenoviral gene therapy vectors.

The above examples are provided to illustrate the invention but not to limit its scope. Other variants of the invention will be readily apparent to one of ordinary skill in the art and are encompassed by the appended claims. All publications, patents, and patent applications cited herein are hereby incorporated by reference for all purposes.

1. A complex comprising:
a) an adenovirus noncovalently complexed to;
b) a copolymer comprising a structure according to Formula I: 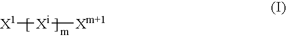
wherein
m is an integer from 1 to 1,000;
i is an integer from 2 to m and denotes the position of Xⁱ;
X¹, Xⁱ, and X^{m+1} are independently selected monomers, wherein
(i) said monomers comprise an amine selected from secondary amines and tertiary amines; and
(ii) at least one of said monomers comprise Q, a structure selected from Formula IIa and Formula IIb: 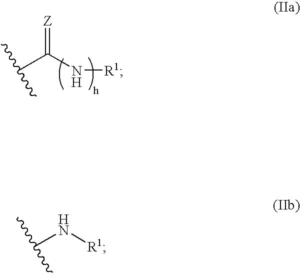
wherein Z is selected from the group consisting of O and NH;
h is an integer from 0 to 1; and
R¹ comprises a polyalkylene glycol moiety;
the copolymer is free of cross-polymerization; and
at physiological pH, at least one of the nitrogen atoms in said copolymer is positively charged.

2. The complex of claim 1, wherein Q is Formula Ia, h is 0, Z is O, and said monomers comprise a structure according to Formula III: 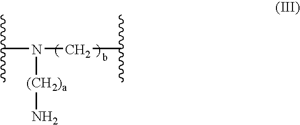
wherein
a is an integer from 1 to 10; and
b is an integer from 1 to 10.

3. The complex of claim 1, wherein Q is Formula Ia, h is 0, Z is O, and said monomers comprise a structure according to Formula IV: 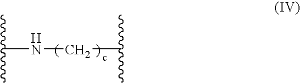
wherein
c is an integer from 1 to 10.

4. The complex of claim 1, wherein Q is Formula Ia, h is 0, Z is O, and said copolymer comprises:
a) at least one of said monomers comprise a structure according to Formula III:
wherein
a is an integer from 1 to 10; and
b is an integer from 1 to 10; and
b) at least one of said monomers comprise a structure according to Formula IV:
wherein
c is an integer from 1 to 10.

5. The complex of claim 4, wherein a is 2.

6. The complex of claim 5, wherein b is 2.

7. The complex of claim 6, wherein c is 2.

8. The complex of claim 4, wherein a is 3.

9. The complex of claim 8, wherein b is 3.

10. The complex of claim 9, wherein c is 3.

11. The complex according to claim 4, wherein said monomer is ethylene imine.

12. The complex of claim 1, wherein Q is Formula Ia, h is 0, Z is O, and said monomers comprise a structure according to Formula V:

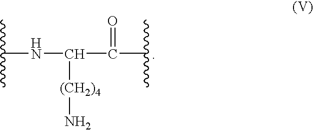

13. The complex of claim 1, wherein said monomer is lysine.

14. The complex of claim 1, wherein Q is Formula Ia, h is 1, Z is NH, and said monomers comprise a structure according to Formula VI: 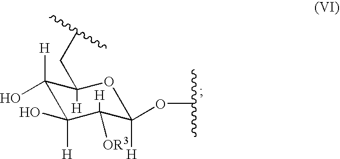
wherein R³ is a member selected from H, —(CH₂CH₂)NH(CH₂CH₃)₂, and —(CH₂CH₂)N(CH₂CH₃)₂CH₂CH₂NH(CH₂CH₃)₂.

15. The complex of claim 14, wherein R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂.

16. The complex of claim 1, wherein said monomer is DEAE-Dextran.

17. The complex of claim 1, wherein Q is Formula IIb, and said copolymer comprises:
a) at least one monomer which comprises a structure according to Formula VI: 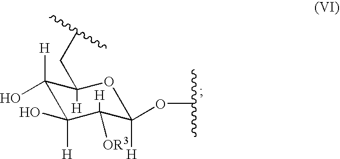
wherein R³ is a member selected from H, —(CH₂CH₂)NH(CH₂CH₃)₂, and —(CH₂CH₂)N(CH₂CH₃)₂CH₂CH₂NH(CH₂CH₃)₂; and
b) at least one monomer which comprises a structure according to Formula VII:

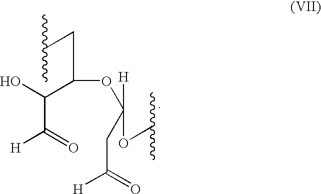

18. The complex of claim 17, wherein R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂.

19. The complex of claim 17, wherein the percentage of said monomers which comprise a structure according to Formula VII is between 5 and 25.

20. The complex of claim 18, wherein the percentage of said monomers which comprise a structure according to Formula VII is between 5 and 25.

21. The complex according to claim 1, wherein R¹ comprises a structure according to Formula VIII: 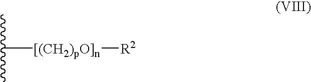
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl.

22. The complex according to claim 21, wherein p is 2.

23. The complex according to claim 4, wherein Q has a structure according to Formula IX: 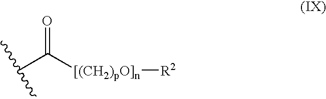
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl.

24. The complex of claim 1, wherein the percentage of monomers which are substituted with Q is at least 10.

25. The complex of claim 1, wherein the percentage of monomers which are substituted with Q is from 15 to 30.

26. The complex according to claim 12, wherein Q has a structure according to Formula IX wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl.

27. The complex according to claim 14, wherein Q has a structure according to Formula X: 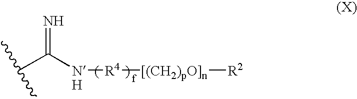
wherein
n is an integer from 2 to 2,000;
p is an integer from 1 to 8;
f is an integer from 0 to 1;
R⁴ has a structure according to Formula XI when f is 1: 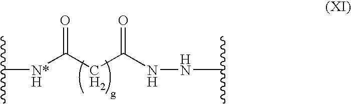
wherein N′ is covalently attached to N*, and wherein g is an integer from 1 to 9;
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl.

28. The complex of claim 27, wherein f is 0.

29. The complex of claim 27, wherein f is 1.

30. The complex of claim 29, wherein g is 4.

31. The complex according to claim 17, wherein Q has a structure according to Formula XII: 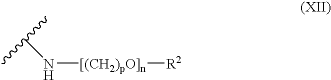
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl.

32. The complex of claim 1, wherein the complex has a diameter of between about 20 nm and about 300 nm.

33. The complex of claim 1, wherein the complex has a diameter of between about 80 nm and about 150 nm.

34. The complex of claim 23, wherein R² is a member selected from substituted and unsubstituted alkyl, substituted and unsubstituted aryl, folate, transferrin, galactose, glucose, antibodies, antibody fragments, and peptides.

35. The complex of claim 34, wherein R² is methyl.

36. The complex of claim 35, wherein p is 2, a is 2, b is 2, and c is 2.

37. The complex of claim 26, wherein R² is a member selected from substituted and unsubstituted alkyl, substituted and unsubstituted aryl, folate, transferrin, galactose, glucose, antibodies, antibody fragments, and peptides.

38. The complex of claim 37, wherein R² is methyl.

39. The complex of claim 38, wherein p is 2.

40. The complex of claim 28, wherein R is a member selected from substituted and unsubstituted alkyl, substituted and unsubstituted aryl, folate, transferrin, galactose, glucose, antibodies, antibody fragments, and peptides.

41. The complex of claim 40, wherein R² is methyl.

42. The complex of claim 41, wherein p is 2.

43. The complex of claim 29, wherein R² is a member selected from substituted and unsubstituted alkyl, substituted and unsubstituted aryl, folate, transferrin, galactose, glucose, antibodies, antibody fragments, and peptides.

44. The complex of claim 44, wherein R² is methyl.

45. The complex of claim 45, wherein p is 2.

46. The complex of claim 30, wherein R² is a member selected from substituted and unsubstituted alkyl, substituted and unsubstituted aryl, folate, transferrin, galactose, glucose, antibodies, antibody fragments, and peptides.

47. The complex of claim 46, wherein R² is methyl.

48. The complex of claim 47, wherein p is 2.

49. The complex of claim 31, wherein R² is a member selected from substituted and unsubstituted alkyl, substituted and unsubstituted aryl, folate, transferrin, galactose, glucose, antibodies, antibody fragments, and peptides.

50. The complex of claim 49, wherein R² is methyl.

51. The complex of claim 50, wherein p is 2.

52. The complex of claim 1, wherein the ratio of the copolymer to the adenovirus is between 3,500:1 and 30,000:1.

53. The complex of claim 52, wherein the ratio of the copolymer to the adenovirus is between 3,600:1 and 20,000:1.

54. The complex of claim 52, wherein the ratio of the copolymer to the adenovirus is between 10,000:1 and 30,000:1.

55. The complex of claim 1, wherein the molecular weight of the copolymer is between 150 kDa and 170 kDa.

56. The complex of claim 1, wherein m is an integer from 10 to 900.

57. The complex of claim 56, wherein m is an integer from 50 to 600.

58. The complex of claim 57, wherein m is an integer from 75 to 300.

59. A method of preparing a noncovalently complexed adenovirus copolymer complex, the method comprising:
contacting a copolymer to an adenovirus, said copolymer comprising a structure according to Formula I wherein
m is an integer from 1 to 1,000;
i is an integer from 2 to m and denotes the position of Xⁱ;
X₁, Xⁱ, and X^{m+1} are independently selected monomers, wherein
(i) said monomers comprise an amine selected from secondary amines and tertiary amines; and
(ii) at least one of said monomers comprise Q, a structure selected from Formula IIa and Formula IIb: 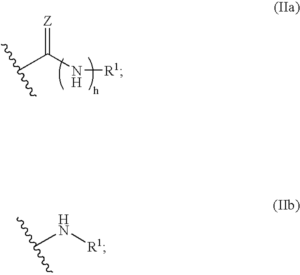
wherein Z is selected from the group consisting of O and NH;
h is an integer from 0 to 1; and
R¹ comprises a polyalkylene glycol moiety;
the copolymer is free of cross-polymerization; and
at physiological pH, at least one of the nitrogen atoms in said copolymer is positively charged.

60. The method of claim 59, wherein Q is Formula IIa, h is 0, Z is O, and said monomers comprise:
a) at least one monomer has a structure according to Formula III wherein
a is an integer from 1 to 10; and
b is an integer from 1 to 10; and
b) at least one monomer has a structure according to Formula IV wherein
c is an integer from 1 to 10;
wherein at least 10% of said monomers are substituted with a structure according to Formula IX
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

61. The method of claim 60, wherein p is 2, a is 2, b is 2, c is 2, and R² is methoxy.

62. The method of claim 59, wherein Q is Formula IIa, h is 0, Z is O, and said monomers comprise a structure according to Formula V:
wherein at least 10% of said monomers are substituted with a structure according to Formula IX
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

63. The method of claim 62, wherein p is 2 and R² is methoxy.

64. The method of claim 59, wherein Q is Formula Ia, h is 1, Z is NH, and said monomers comprise a structure according to Formula VI;
wherein R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂,
wherein the percentage of monomers which are substituted with Q is at least 10 and Q has a structure according to Formula X;
wherein
n is an integer from 2 to 2,000;
p is an integer from 1 to 8;
f is 0; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

65. The method of claim 64, wherein p is 2 and R² is methoxy.

66. The method of claim 59, wherein Q is Formula IIa, h is 1, Z is NH, and said monomers comprise a structure according to Formula VI;
wherein R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂,
wherein the percentage of monomers which are substituted with Q is at least 10 and Q has a structure according to Formula X;
wherein
n is an integer from 2 to 2,000;
p is an integer from 1 to 8;
f is 1;
R⁴ has a structure according to Formula XI;
g is 4; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

67. The method of claim 66, wherein p is 2 and R² is methoxy.

68. The method of claim 59, wherein Q is Formula IIb, and said copolymer comprises:
a) at least one monomer which comprises a structure according to Formula VI;
wherein R³ is a member selected from H, —(CH₂CH₂)NH(CH₂CH₃)₂, and —(CH₂CH₂)N(CH₂CH₃)₂CH₂CH₂NH(CH₂CH₃)₂; and
b) at least one monomer which comprises a structure according to Formula VII;
wherein the percentage of monomers which are substituted with Q is at least 10 and Q has a structure according to Formula XII;
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

69. The method of claim 68, wherein p is 2 and R² is methoxy.

70. A method of introducing an adenovirus into a cell, the method comprising:
(a) noncovalently contacting a copolymer to an adenovirus, said copolymer comprising a structure according to Formula I wherein
m is an integer from 1 to 1,000;
i is an integer from 2 to m and denotes the position of Xⁱ;
X¹, Xⁱ, and X^{m+1} are independently selected monomers, wherein
(i) said monomers comprise an amine selected from secondary amines and tertiary amines; and
(ii) at least one of said monomers comprise Q, a structure selected from Formula Ia and Formula IIb: 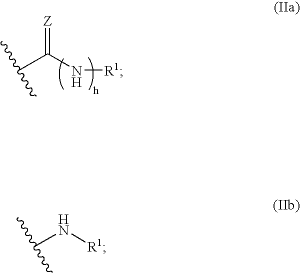
wherein Z is selected from the group consisting of O and NH;
h is an integer from 0 to 1; and
R¹ comprises a polyalkylene glycol moiety;
the copolymer is free of cross-polymerization; and
at physiological pH, at least one of the nitrogen atoms in said copolymer is positively charged; and
(b) contacting the complex to a cell.

71. The method of claim 70, wherein the adenovirus is more infective than the adenovirus without the copolymer.

72. The method of claim 71, wherein the adenovirus is at least 20% more infective than without the copolymer.

73. The method of claim 71, wherein the adenovirus is at least 100% more infective than without the copolymer.

74. The method of claim 70, wherein Q is Formula Ia, h is 0, Z is O, and said monomers comprise:
a) at least one monomer has a structure according to Formula III wherein
a is an integer from 1 to 10; and
b is an integer from 1 to 10; and
b) at least one monomer has a structure according to Formula IV wherein
c is an integer from 1 to 10;
wherein at least 10% of said monomers are substituted with a structure according to Formula IX
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

75. The method of claim 74, wherein p is 2, a is 2, b is 2, c is 2, and R² is methoxy.

76. The method of claim 70, wherein Q is Formula Ia, h is 0, Z is O, and said monomers comprise a structure according to Formula V:
wherein at least 10% of said monomers are substituted with a structure according to Formula IX
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

77. The method of claim 76, wherein p is 2 and R² is methoxy.

78. The method of claim 70, wherein Q is Formula IIa, h is 1, Z is NH, and said monomers comprise a structure according to Formula VI;
wherein R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂,
wherein the percentage of monomers which are substituted with Q is at least 10 and Q has a structure according to Formula X;
wherein
n is an integer from 2 to 2,000;
p is an integer from 1 to 8;
f is 0; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

79. The method of claim 78, wherein p is 2 and R² is methoxy.

80. The method of claim 70, wherein Q is Formula Ia, h is 1, Z is NH, and said monomers comprise a structure according to Formula VI;
wherein R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂,
wherein the percentage of monomers which are substituted with Q is at least 10 and Q has a structure according to Formula X;
wherein
n is an integer from 2 to 2,000;
p is an integer from 1 to 8;
f is 1;
R⁴ has a structure according to Formula XI;
g is 4; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

81. The method of claim 80, wherein p is 2 and R² is methoxy.

82. The method of claim 70, wherein Q is Formula IIb, and said copolymer comprises:
a) at least one monomer which comprises a structure according to Formula VI;
wherein R³ is a member selected from H, —(CH₂CH₂)NH(CH₂CH₃)₂, and —(CH₂CH₂)N(CH₂CH₃)₂CH₂CH₂NH(CH₂CH₃)₂; and
b) at least one monomer which comprises a structure according to Formula VII;
wherein the percentage of monomers which are substituted with Q is at least 10 and Q has a structure according to Formula XII;
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

83. The method of claim 82, wherein p is 2 and R² is methoxy.

84. A physiological formulation comprising:
(a) a copolymer comprising:
a structure according to Formula I wherein
m is an integer from 1 to 1,000;
i is an integer from 2 to m and denotes the position of Xⁱ;
X¹, Xⁱ, and X^{m+1} are independently selected monomers, wherein
(i) said monomers comprise an amine selected from secondary amines and tertiary amines; and
(ii) at least one of said monomers comprise Q, a structure selected from Formula Ia and Formula IIb: 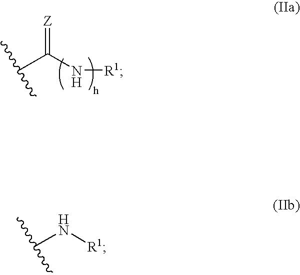
wherein Z is selected from the group consisting of O and NH;
h is an integer from 0 to 1; and
R¹ comprises a polyalkylene glycol moiety;
the copolymer is free of cross-polymerization; and
at physiological pH, at least one of the nitrogen atoms in said copolymer is positively charged;
(b) an adenovirus, wherein the copolymer forms a noncovalent complex with the adenovirus; and
(c) a physiologically acceptable excipient.

85. The physiological formulation of claim 84, wherein Q is Formula Ia, h is 0, Z is O, and said monomers comprise:
a) at least one monomer has a structure according to Formula III wherein
a is an integer from 1 to 10; and
b is an integer from 1 to 10; and
b) at least one monomer has a structure according to Formula IV wherein
c is an integer from 1 to 10;
wherein at least 10% of said monomers are substituted with a structure according to Formula IX
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

86. The physiological formulation of claim 85, wherein p is 2, a is 2, b is 2, c is 2, and R² is methoxy.

87. The physiological formulation of claim 84, wherein Q is Formula IIa, h is 0, Z is O, and said monomers comprise a structure according to Formula V:
wherein at least 10% of said monomers are substituted with a structure according to Formula IX
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

88. The physiological formulation of claim 87, wherein p is 2 and R² is methoxy.

89. The physiological formulation of claim 84, wherein Q is Formula Ia, h is 1, Z is NH, and said monomers comprise a structure according to Formula VI;
wherein R³ is H for about two of every three of said monomers, and for about one of every three of said monomers, R is a member selected from —(CH₂CH₂)NH(CH₂CH₃)₂, and —(CH₂CH₂)N(CH₂CH₃)₂CH₂CH₂NH(CH₂CH₃)₂,
wherein the percentage of monomers which are substituted with Q is at least 10 and Q has a structure according to Formula X;
wherein
n is an integer from 2 to 2,000;
p is an integer from 1 to 8;
f is 0; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

90. The physiological formulation of claim 89, wherein p is 2 and R² is methoxy.

91. The physiological formulation of claim 84, wherein Q is Formula Ia, h is 1, Z is NH, and said monomers comprise a structure according to Formula VI;
wherein R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂,
wherein the percentage of monomers which are substituted with Q is at least 10 and Q has a structure according to Formula X;
wherein
n is an integer from 2 to 2,000;
p is an integer from 1 to 8;
f is 1;
R⁴ has a structure according to Formula XI;
g is 4; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

92. The physiological formulation of claim 91, wherein p is 2 and R² is methoxy.

93. The physiological formulation of claim 84, wherein Q is Formula IIb, and said copolymer comprises:
a) at least one monomer which comprises a structure according to Formula VI;
wherein R³ is a member selected from H, —(CH₂CH₂)NH(CH₂CH₃)₂, and —(CH₂CH₂)N(CH₂CH₃)₂CH₂CH₂NH(CH₂CH₃)₂; and
b) at least one monomer which comprises a structure according to Formula VII;
wherein the percentage of monomers which are substituted with Q is at least 10 and Q has a structure according to Formula XII;
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

94. The physiological formulation of claim 93, wherein p is 2 and R² is methoxy.

95. A kit comprising a copolymer and an adenovirus, wherein the copolymer and adenovirus are noncovalently attached,
said copolymer comprising:
a structure according to Formula I wherein
m is an integer from 1 to 1,000;
i is an integer from 2 to m and denotes the position of Xⁱ;
X¹, Xⁱ, and X^{m+1} are independently selected monomers, wherein
(i) said monomers comprise an amine selected from secondary amines and tertiary amines; and
(ii) at least one of said monomers comprise Q, a structure selected from Formula IIa and Formula IIb: 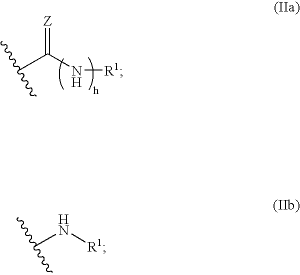
wherein Z is selected from the group consisting of O and NH;
h is an integer from 0 to 1; and
R¹ comprises a polyalkylene glycol moiety;
the copolymer is free of cross-polymerization; and
at physiological pH, at least one of the nitrogen atoms in said copolymer is positively charged.

96. The kit of claim 95, wherein Q is Formula IIa, h is 0, Z is O, and said monomers comprise:
a) at least one monomer has a structure according to Formula III wherein
a is an integer from 1 to 10; and
b is an integer from 1 to 10; and
b) at least one monomer has a structure according to Formula 1V wherein
c is an integer from 1 to 10;
wherein at least 10% of said monomers are substituted with a structure according to Formula IX
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

97. The kit of claim 96, wherein p is 2, a is 2, b is 2, c is 2, and R² is methoxy.

98. The kit of claim 95, wherein Q is Formula IIa, h is 0, Z is O, and said monomers comprise a structure according to Formula V:
wherein at least 10% of said monomers are substituted with a structure according to Formula IX;
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

99. The kit of claim 98, wherein p is 2 and R² is methoxy.

100. The kit of claim 95, wherein Q is Formula Ia, h is 1, Z is NH, and said monomers comprise a structure according to Formula VI;
wherein R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂,
wherein the percentage of monomers which are substituted with Q is at least 10 and Q has a structure according to Formula X;
wherein
n is an integer from 2 to 2,000;
p is an integer from 1 to 8;
f is 0; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

101. The kit of claim 100, wherein p is 2 and R² is methoxy.

102. The kit of claim 95, wherein Q is Formula Ia, h is 1, Z is NH, and said monomers comprise a structure according to Formula VI;
wherein R³ is H for about two of every three of said monomers, and for about one of every three of said monomers, R³ is a member selected from —(CH₂CH₂)NH(CH₂CH₃)₂, and —(CH₂CH₂)N(CH₂CH₃)₂CH₂CH₂NH(CH₂CH₃)₂,
wherein the percentage of monomers which are substituted with Q is at least 10 and Q has a structure according to Formula X;
wherein
n is an integer from 2 to 2,000;
p is an integer from 1 to 8;
f is 1;
R⁴ has a structure according to Formula XI;
g is 4; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

103. The kit of claim 102, wherein p is 2 and R² is methoxy.

104. The kit of claim 95, wherein Q is Formula IIb, and said copolymer comprises:
a) at least one monomer which comprises a structure according to Formula VI;
wherein R³ is a member selected from H, —(CH₂CH₂)NH(CH₂CH₃)₂, and —(CH₂CH₂)N(CH₂CH₃)₂CH₂CH₂NH(CH₂CH₃)₂; and
b) at least one monomer which comprises a structure according to Formula VII;
wherein the percentage of monomers which are substituted with Q is at least 10 and Q has a structure according to Formula XII;
wherein
n is an integer from 2 to 2,000;
p is an integer from 1 to 8; and
R² is a member selected from substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl;
the copolymer is free of cross-polymerization; and
at physiological pH, at least one of the nitrogen atoms in said copolymer is positively charged.

105. The kit of claim 104, wherein p is 2 and R² is methoxy.


SEQUENCE LISTING (6 sequences)

SEQ ID NO: 1 - DNA, 17 bp, Artificial Sequence
  Description of Artificial SequencePCR BGCG forward primer
aacggtactccgccacc

SEQ ID NO: 2 - DNA, 22 bp, Artificial Sequence
  Description of Artificial SequencePCR BGCG reverse primer
actggttagacgcctttctcga

SEQ ID NO: 3 - DNA, 19 bp, Artificial Sequence
  Description of Artificial SequenceBGCG probe
nccgcatcgaccggatcgn

SEQ ID NO: 4 - DNA, 18 bp, Artificial Sequence
  Description of Artificial SequencePCR GAPDH forward primer
gaaggtgaaggtcgagtc

SEQ ID NO: 5 - DNA, 20 bp, Artificial Sequence
  Description of Artificial SequencePCR GAPDH reverse primer
gaagatggtgatgggatttc

SEQ ID NO: 6 - DNA, 20 bp, Artificial Sequence
  Description of Artificial SequenceGAPDH probe
naagcttcccgttctcagcn